(19) United States
(12) Patent Application Publication
(10) Pub. No.: US 2002/0001610 A1
(43) Pub. Date: Jan. 3, 2002

(54) Active ingredient delivery systems and devices based on porous matrices

(76) Inventors: Smadar Cohen, Beer Sheva (IL); Lilia Shapiro, Beer Sheva (IL); Amnon Sintov, Omer (IL)

Correspondence Address: Gregory A. Sebald, MERCHANT & GOULD P.C., P.O. Box 2903, Minneapolis, MN, 55402-0903, US

(21) Appl. No.: 09/737,665

(22) Filed: Dec. 15, 2000

Related U.S. Application Data
(63) Continuation-in-part of application No. PCT/IL99/00327, filed on Jun. 15, 1999.

(30) Foreign Application Priority Data
Jun. 16, 1998 (IL) 124957

(51) Int. Cl. A61K 9/64; A61K 9/14

(52) U.S. Cl. 424/439; 424/484

(57) ABSTRACT

The invention is primarily directed to a mucosal delivery system comprising a porous matrix incorporating an active ingredient, said matrix have a pore size distribution such as to promote adhesion to a mucosal surface, and to permit transfer of said active ingredient toward the mucosal surface.

### FIELD OF THE INVENTION

The present invention is concerned with the use of porous matrices, particularly polysaccharide sponges, as devices for the delivery of drugs to mucosal surfaces as well as to the luminal fluid of the gastrointestinal tract.

### BACKGROUND OF THE INVENTION

The goal of any drug delivery system is to provide a therapeutic amount of drug to the proper site in the body, in order to achieve and then maintain the desired drug concentration. In the case of many drugs, particularly those designed for use in the management of chronic diseases, there is a requirement for a drug delivery system that is able to administer a controlled release of the therapeutic agent for periods ranging from several days to several years. The drug-bearing implant or similar device is one form of delivery system that in theory is well suited to such requirements. Implants for drug-delivery may be used to deliver therapeutic agents into various different tissues and body cavities including the skin surface, subcutaneous tissue, the eye and the uterus. For some agents, however, pharmacokinetic considerations dictate that mucosal surfaces (such as in the mouth, intestine, reproductive tract etc.) are the preferred delivery site. The key factor in the design of such a system is the selection of the appropriate polymeric or other material, that will enable the optimal delivery of a drug to the chosen body site. In particular, a desirable feature of such a delivery system would be the possibility to produce it in an ingestible form, in order to bring it into contact with a required region of the gastrointestinal tract. In addition to biocompatibility, other desirable features of the material include biodegradation rates, hydrophobicity/hydrophilicity and pore size. Examples of materials that have been previously used for therapeutic implants and related devices include silicones, polyethylenes and ethylene-vinyl acetate copolymers (Gennaro, A. R., Remington's Pharmaceutical Sciences, 18th ed., 1990, pp. 1688-1689).

Porous, absorbable matrices fabricated from natural and synthetic polymers (see, for example, Langer R. and Vacanti J. P., Science 260: 920-926, 1993), are currently in use or under investigation as implants, for use in a variety of applications including the facilitation of tissue regeneration in defects caused by disease, trauma or reconstructive surgical procedures. A copending PCT application, WO 97/44070, the specification of which is incorporated herein by reference, describes a method of preparation of bioresorbable polysaccharide sponges, and their use as in vitro and in vivo cell cultivation matrices. Such sponges are examples of suitable matrices for the delivery system of the invention.

The macromolecular structure of the sponges described in the aforementioned PCT application is selected so that they are completely degradable and are eliminated, once they have achieved their function. Thus, by careful selection of sponges with particular macromolecular conformations, it is possible to produce porous matrices with a desired life time of activity.

Most of the porous matrices developed to date are based on natural polymers such as collagen, or synthetic polymers from the lactic/glycolic acid family. The collagen-based matrices have several disadvantages, including: they degrade at relatively rapid rate; many disappearing as early as 4 weeks postimplantation (Ben-Yishay, A. et al., Tissue Engineering 1: 119-132, 1995). Although the rate of degradation of the collagen matrix may be reduced by cross-linking with glutaraldehyde, the resulting cross-linked matrices, however, exhibited immunogenicity, calcification, and fibrous scarring when implanted for long periods.

Other synthetic biodegradable foams based on poly(D, L-Lactic-co glycolic acid) have been developed as scaffolds for tissue engineering, as noted above, but because these polymers are hydrophobic when a liquid media is placed on these foams or injected into their interior, the majority of their pores remain empty, resulting in the underutilization of the volume of these foams. In addition, studies have also shown that the degradation of these biodegradable foams results in the significant accumulation of acid products which significantly decreases the internal pH within the foam to less than pH 3.0 (see Park Lu and Crotts, Journal of Controlled Release 33: 211-222, 1995), which acidity is very harmful to living tissue.

Alginates have also been used previously as implants for the purpose of cell transplantation. Alginates are natural polysaccharide polymers, the word “alginate” actually referring to a family of polyanionic polysaccharide copolymers derived from brown sea algae and comprising 1,4-linked β-D-mannuronic (M) and α-L-guluronic acid (G) residues in varying proportions. Alginate is soluble in aqueous solutions, at room temperature, and is capable of forming stable gels, particularly in the presence of certain divalent cations such as calcium, barium, and strontium. The unique properties of alginate, together with its biocompatibility (see Sennerby et al., 1987 and Cohen et al., 1991), its relatively low cost and wide availability have made alginate an important polymer in medicinal and pharmaceutical applications. For example, it has been used in wound dressings and dental impression materials. Further, alginate has also been approved by various regulatory authorities as acceptable for use as a wound dressing and as food additives in humans. Moreover, pharmaceutical grade alginates, which comply with all the quality and safety requirements of the European and United States of America (USA) pharmacological regulatory authorities, are readily available from several commercial manufacturers.

### SUMMARY OF THE INVENTION

It has now been surprisingly found that porous polymeric matrices have an additional and unexpected property of facilitating the entry of regions of the mucous membrane to which the matrix is attached, into the pores and internal channels of said matrices. In this way, projections of mucosa are enclosed by the pores of the matrix, permitting extremely close contact of these projections with the liquid contents of the matrix interior, allowing highly efficient absorption of drugs and other agents by the enclosed mucous membrane. The area of mucosal surface in direct contact with the agents held by the matrix is determined only by the porosity of the polymeric material of which the matrix is made, and in any event is significantly greater than that achieved with previously known types of drug-delivery device.

The invention is primarily directed to the provision of a luminal and mucosal delivery system comprising a porous matrix incorporating a therapeutic agent, said matrix having a pore size distribution such as to promote adhesion thereof to a mucosal surface, and to permit transfer of said therapeutic agent toward the mucosal surface, as well as into the luminal fluid of the gastrointestinal tract.

Throughout this specification, wherever reference is made to therapeutic agents, it is understood that all kinds of active ingredients, such as food supplements, are also meant, reference being made in the description to therapeutic agents only for the sake of brevity.

Although many types of material may be used to construct the porous matrix delivery system of the present invention, a preferred matrix has the following physical parameters:

i. an average pore size in the range of about 10 μm to about 300 μm;

ii. an average distance between the pores being the wall thickness of the pores in the range of about 5 μm to about 270 μm;

iii. an E-modulus of elasticity in the range of about 50 kPa to about 500 kPa.

A stated above, a variety of materials may be used to construct the delivery system of the invention. According to a preferred embodiment of the invention, however, the porous matrix comprises a polysaccharide sponge.

One aspect of the invention is directed to the use of- the mucosal delivery system, wherein the mucosal surface is the intestinal mucosa.

In another aspect, the invention is directed to a delivery system for the delivery of therapeutic agents to the gastric mucosa.

In a further aspect, the invention is directed to a delivery system for the delivery of therapeutic agents into the luminal fluid of the gastrointestinal tract.

In still a further aspect, the delivery system of the invention is used to deliver therapeutic agents to the oral mucosa.

As described above, the porous matrix of the drug-delivery device, in a preferred embodiment, may comprise a polysaccharide sponge. This polysaccharide sponge may be produced in various different physical forms for use in this invention. In one preferred embodiment, the polysaccharide sponge is in the form of bioadhesive sponge-based macrospheres, said macrospheres having a size distribution of 100-1000 μm.

In another aspect, the invention is directed towards the use of polysaccharide sponges in the form of bioadhesive sponge-based cylindrical matrices.

The invention also encompasses a mucosal delivery system wherein the bioadhesive material is in the form of a bioadhesive core which is coated with one or more active or inert coating layers. One purpose of the said coating layers is to permit the use of the delivery system to deliver therapeutic agents to different portions of the intestinal lumen or mucosa along the gastrointestinal tract, the exact location being determined by the type of coating, following ingestion of the coated device. In one embodiment, the coating layer may comprise a capsule. A preferred type of capsule for use in this aspect of the invention is a gelatine capsule. According to another preferred embodiment, the aforementioned coating layer comprises an enteric coating.

According to still another preferred embodiment, the aforementioned coating layer comprises a coating intended for colonic targeting.

In a further aspect, the invention is directed to the use of a porous matrix, as described above, in the preparation of a drug delivery device for use at mucosal surfaces.

In a still further aspect, the invention is directed to the use of a porous matrix in the preparation of a drug delivery device for the delivery of insulin to mucosal surfaces.

The invention is further -directed to a polysaccharide sponge, for use as a mucosal drug delivery device.

All the above and other characteristics and advantages of the invention will be further understood from the following illustrative and non-limitative examples of preferred embodiments thereof.

### BRIEF DESCRIPTION OF THE DRAWINGS

The present invention will be more clearly understood from the detailed description of the preferred embodiments and from the attached drawings in which:

FIG. 1 is a line graph depicting the change in plasma glucose levels following the insertion of 12 mg of insulin-loaded bioadhesive sponge-based macrospheres (BSMS) into the duodenum of Sprague-Dawley rats. The total amount of insulin given to each animal was 10 units.

FIG. 2 is a line graph depicting the plasma glucose levels following the implantation of 12 mg of bioadhesive sponge-based cylindrical matrix (BSCM) (not containing insulin) into the duodenum of Sprague -Dawley rats.

FIG. 3 is a line graph depicting the change in plasma glucose levels following the insertion of 12 mg of insulin-loaded bioadhesive sponge-based cylindrical matrices (BSCM) into the duodenum of Sprague-Dawley rats. The total amount of insulin given to each animal was 10 units.

FIG. 4 is a line graph depicting the changes in plasma glucose levels following the insertion of 12 mg of insulin-loaded bioadhesive sponge-based macrospheres (BSMS) into either the jejunum or ileum of Sprague-Dawley rats. The total amount of insulin given to each animal was 10 units.

### DETAILED DESCRIPTION OF PREFERRED EMBODIMENTS

### Methods

Composition of bioadhesive devices

The drug-delivery devices of this invention may comprise any suitable porous material. The following list of illustrative and non-limitative examples of such materials used in the manufacture of polysaccharide sponges is given for illustrative purposes, and is not intended to limit the scope of the invention in any way:

Polyanionic polysaccharides

Alginates, Gellan, Gellan gum, Xanthan, Agar, Carrageenan;

Cationic polysaccharides

Chitosan.

Composition of alginate sponges

For the sake of brevity, the invention will be illustrated hereinafter using alginate sponges as the bioadhesive materials, it being understood that such examples are provided for the purpose of illustration, and that any other suitable bioadhesive material can be substituted. The alginate sponges described in the examples that follow may be manufactured from the following alginate types, but not limited to these types alone:

| | Guluronic | Viscosity | |
|---|---|---|---|
| Alginate ™ | acid (%) | (1%, 25° C.) | Algae |
| MVG | 70 | 200-800 | Laminaria hyperborea |
| | | | (stem) |
| Protanal HF 120 | 65-75 | 600-800 | Laminaria hyperborea |
| | | | (stem) |
| Protanal SF 120 | 65-75 | 400-600 | Laminaria hyperborea |
| | | | (stem) |
| Protanal SF 120 RB | 35-45 | 400-600 | Laminaria hyperborea |
| | | | (leaves) |
| Protanal LF 20/60 | 65-75 | 100-150 | Laminaria hyperborea |
| | | | (stem) |
| Protanal LF 120 | 65-75 | 200-400 | Laminaria hyperborea |
| | | | (stem) |
| Protanal LF 200 RB | 40-50 | 200-400 | Laminaria hyperborea |
| | | | (leaves) |
| Keltone HVCR | 39 | 400 | Macrocystis pyrifera |
| Manugeel DMB | 69 | 200-400 | Laminaria hyperboria |
| Keltone LV | 39 | 50-150 | Macrocystis pyrifera |

Appropriate cross-linking agents are selected from a group consisting of the salts of calcium, copper, aluminium, magnesium, strontium, barium, tin, zinc, chromium, organic cations, poly(amino acids), poly(ethyleneimine), poly(vinylamine), poly(allyl amine) and polysaccharides. These cross-linking agents are used at a final concentration of 0.1-2.0% (w/v).

### EXAMPLE 1

### Preparation of Alginate Sponge-Based Cylinders

The technology for sponge preparation is based on 3 steps: gelation of alginate solution to form a cross-linked hydrogel, then freezing, and finally drying by lyophilization. Briefly, 0.5-1 ml of alginate solution, 2% w/v, were poured into the wells of a 24-well plate (well size: 16 mm diameter, 20 mm height), diluted to the desired final concentration with double distilled water, and then crosslinked to form a gel by adding from the cross linker solution very slowly, while stirring intensively using the homogenizer (Dispenser tool 6G at speed of 31,800 RPM) for 3 min.

The alginate gels are then frozen. We used two sets of conditions to examine the speed of freezing on sponge morphology and mechanical properties: by placing the plates 1) on a shelf in a freezer at −180 C., or 2) in a liquid nitrogen bath for 15 min. Finally, frozen gels are lyophilized (Freeze Dry systems LABCONCO Co., Kansas City) at 0.007 mm Hg and freeze-drying temperature −60° C.

### EXAMPLE 2

### Preparation of Alginate Sponge-Based Macrospheres

Sodium alginate solution (1% w/v) is prepared by dissolving the polysaccharide in double-distilled water while stirring, followed by filtration of the solution through a series of 1.2, 0.45 and 0.2 μ pore-size membrane filters.

Macrospheres are made by spraying the alginate solution as macrodroplets, using an air jet-head droplet forming apparatus. With this system, the alginate solution is extruded (at 1 ml/min) through a 22G needle located inside a tube through which air flows at 3 l/min. Droplets forming at the needle tip are forced off by the coaxial air stream into the gelation bath. The gelation bath is composed of calcium chloride, 1.5% w/v, pH 7.4. Upon contact of the alginate macrodroplets with the gelation solution, they are gelled and left for hardening for additional 30 min. Macrospheres are collected after draining the calcium chloride solution using a 10 ml filter-ended Econo-column (BioRad).

The concentrated macrospheres are frozen in liquid nitrogen and then lyophilized over night (−60° C., 0.0007 mm Hg).

### EXAMPLE 3

### In-Vivo Evaluation of Insulin-Loaded Bioadhesive Sponge-Based

### Macrospheres (BSMS) upon Administration into the Duodenum

The experimental procedure was performed according to Sintov et al. (Int. J. Pharm. 143: 101-106, 1996). In brief, fasted locally-grown Sprague-Dawley rats were anaesthetized by pentobarbital injection, followed by isolation of their stomach (the pylorus was ligated). A 2 mm incision was made in the duodenum, through which 12 mg of BSMS containing insulin (10 units), were immediately administered. After administration, the duodenum was ligated underneath the incision.

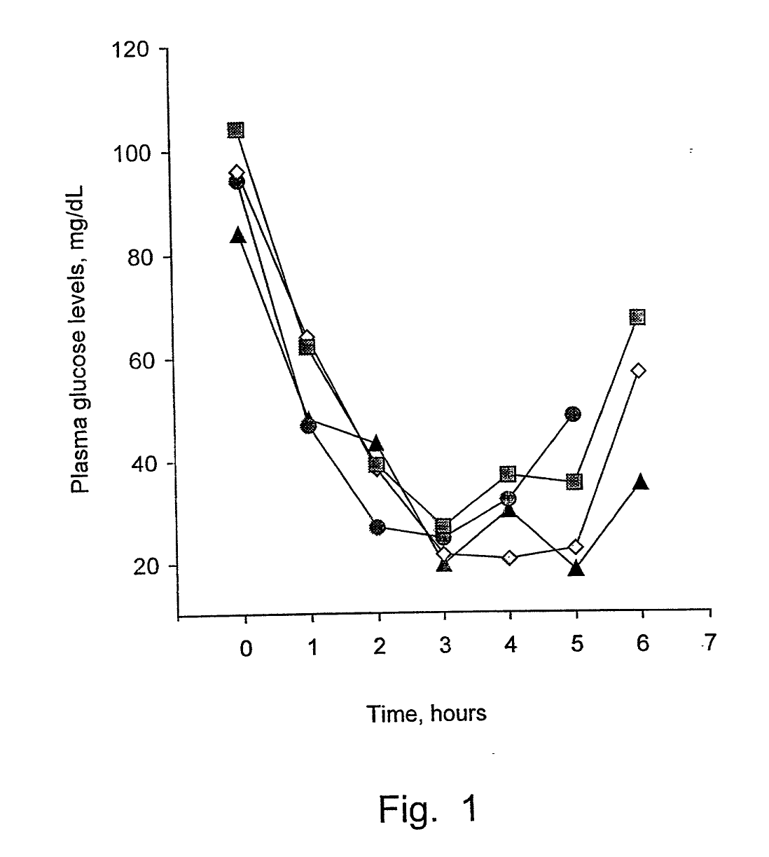

Peripheral plasma (tail vein) was measured for glucose by using a GOD/PAP reagent (Glucose PAP kit, Hoffman-La Roche, Basel, Switzerland), and a spectrophotometric reading at 500 nm wavelength. The results of the glucose assay are shown in FIG. 1.

### EXAMPLE 4

### In-Vivo Evaluation of Insulin-Loaded Bioadhesive Sponge-Based

### Cylindrical Matrices (BSCM) upon Administration into the

### Duodenum

The experimental procedure was performed as described in Example 3, except that 12 mg of BSCM, containing either 10 units of insulin or control medium were introduced into the duodenum.

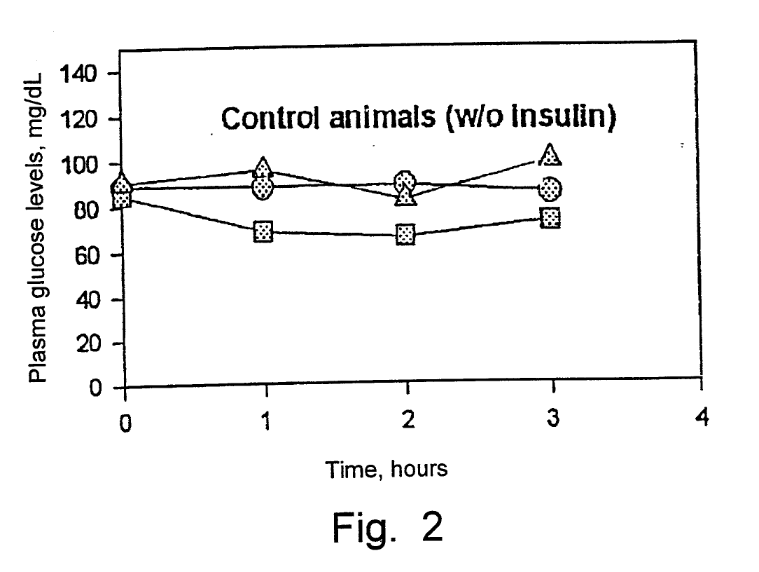

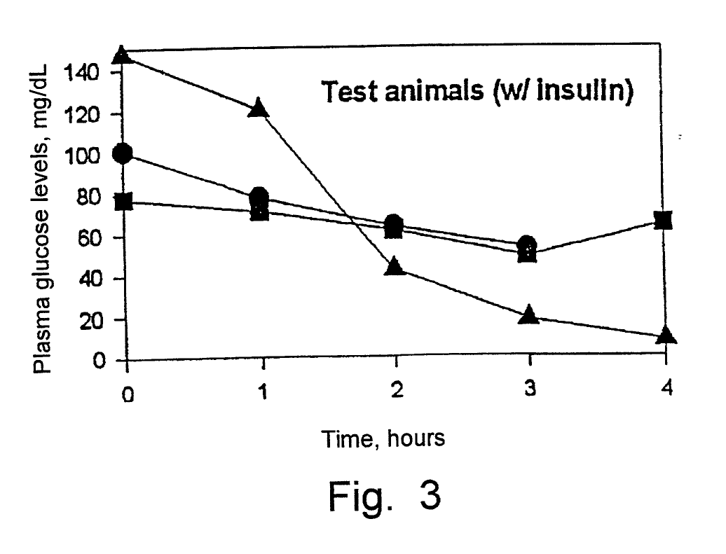

The results of the glucose assays are given in FIG. 2 (control) and FIG. 3 (insulin).

### EXAMPLE 5

### In-Vivo Evaluation of Insulin-Loaded Bioadhesive Sponge-Based

### Macrospheres (BSMS) upon Administration into the Jejunum and

### Ileum

The experimental procedure was performed as described in Example 3, except that the BSMS was introduced into the jejunum in 8 rats, and into the ileum in 4 other rats. Two control experiments were performed, one included administration of insulin without the macrospheres (non-BSMS) to 4 rats, and the other included intraperitoneal (i.p.) administration of 1 unit of insulin to a rat. The latter control was used to demonstrate the parenteral effect produced in this animal species (200-300 g) with the highest non-lethal dose of insulin.

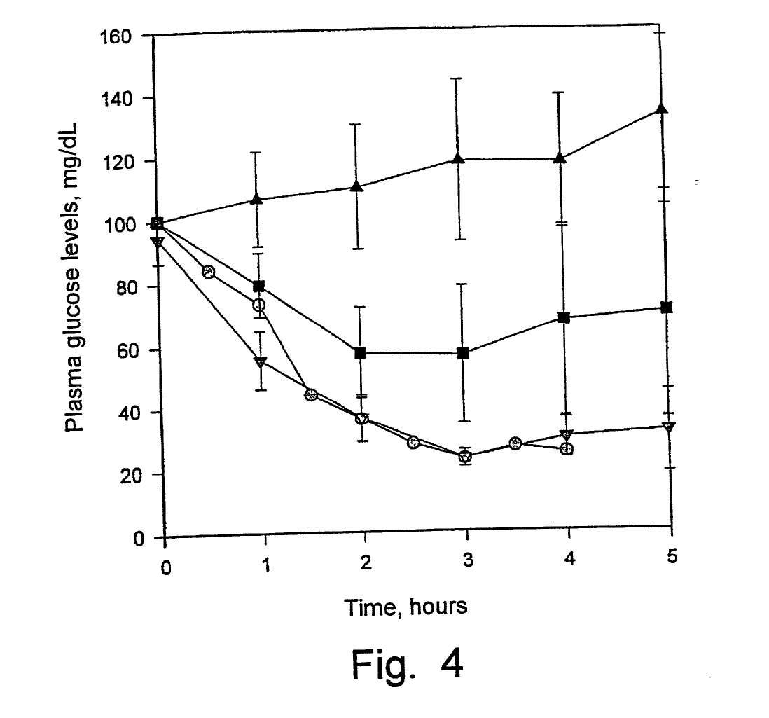

The results of the hypoglycemic effects at the various administrations are presented in FIG. 4. The data points for the jejunal adminstration of insulin (with macrospheres) are shown in FIG. 4 as closed squares. The jejunal administration control (with insulin, without macrospheres) is represented by upward pointing triangles. The ileal administration data points are shown as downward pointing triangles, while the intraperitoneal control is represented by closed circles.

A preliminary pharmacokinetic monitoring of insulin demonstrated 15, 65, 34 and 27 μunits/ml insulin in plasma at 0, 1, 2 and 3 hours after administration, respectively. These increasing values of insulin in rat plasma are parallel to the increasing hypoglycemic effect observed after the ileal insulin-macrosphere administration.

All of the above descriptions and examples have been provided for the purpose of illustration, and are not intended to limit the invention in any way. Many different bioadhesive materials can be used to provide different delivery devices, to deliver various therapeutic agents to different mucosal surfaces, all without exceeding the scope of the invention.

1. A mucosal delivery system comprising a porous matrix incorporating an active ingredient, said matrix having a pore size distribution such as to promote adhesion thereof to a mucosal surface, and to permit transfer of said active ingredient toward the mucosal surface.

2. A luminal delivery system comprising a porous matrix incorporating an active ingredient, said matrix having a pore size distribution such as to promote adhesion thereof to a mucosal surface, and to permit transfer of said active ingredient toward the surrounding fluid thereof.

3. A delivery system according to claims 1 or 2, wherein the active ingredient is a therapeutic agent.

4. A delivery system according to claims 1 or 2, wherein the active ingredient is a food supplement.

5. A delivery system according to any of claims 1 to 4, wherein the matrix has the following physical parameters:
i. an average pore size in the range of about 10 μm to about 300 μm;
ii. an average distance between the pores being the wall thickness of the pores in the range of about 5 μm to about 270 μm;
iii. an E-modulus of elasticity in the range of about 50 kPa to about 500 kPa.

6. A delivery system, according to any one of claims 1 to 5, wherein the porous matrix comprises a polysaccharide sponge.

7. A delivery system, according to any one of claims 1 to 6, wherein the mucosal surface is the intestinal mucosa.

8. A delivery system, according to any ones of claims 1 to 6, wherein the mucosal surface is the gastric mucosa.

9. A delivery system, according to any one of claims 1 to 6, wherein the mucosal surface is the oral mucosa.

10. A polysaccharide sponge for use as a drug-delivery device, wherein the sponge is in the form of bioadhesive sponge-based macrospheres, said macrospheres having a size distribution of 100-1000 μm.

11. A polysaccharide sponge for use as a drug-delivery device, wherein the sponge is in the form of bioadhesive sponge-based cylindrical matrices.

12. A delivery system according to any one of claims 1 to 9, which comprises a bioadhesive core which is coated with one or more active or inert coating layers.

13. A delivery system according to claim 12, wherein the coating comprises a capsule.

14. A drug delivery system according to claim 13, wherein the capsule is a gelatine capsule.

15. A drug delivery system according to any one of claims 12 to 14, wherein the coating comprises an enteric coating.

16. A drug delivery system according to any one of claims 12 to 14, wherein the coating comprises a coating for colonic targeting.

17. The use of a porous matrix as claimed in claim 5, in the preparation of a drug delivery device for use at mucosal surfaces.

18. The use of a porous matrix as claimed in claim 5, in the preparation of a drug delivery device for the delivery of insulin to mucosal surfaces.

19. A polysaccharide sponge, for use as a mucosal drug delivery device.
